(19) United States
(12) Patent Application Publication
(10) Pub. No.: US 2012/0003602 A1
(43) Pub. Date: Jan. 5, 2012

(54) DEVICE FOR OBSERVATION OF THE ORAL CAVITY

(76) Inventors: Michael Waldmeier, Frankfurt/Main (DE); Werner Dalitz, Bad Nauheim (DE); Thorsten Koch, Schwalbach/Taunus (DE); Holger Port, Schwalbach/Taunus (DE)

(21) Appl. No.: 12/828,656

(22) Filed: Jul. 1, 2010

(51) Int. Cl. A61B 1/247

(52) U.S. Cl. 433/30

(57) ABSTRACT

A device for observation of the oral cavity having a mirror arrangement with at least one mirror element assigned to a certain observation zone in the oral cavity area and a positioning structure for positioning the mirror arrangement in the oral cavity in a reproducible manner.

### FIELD OF THE INVENTION

The invention is directed to a device for enabling observation of the oral cavity of a person to be examined. The invention is directed in particular to a device for examining the dental substance as well as optionally a patient's periodontal condition, e.g., to evaluate the hygiene results, the progress of a treatment or other properties of the dental substance or of the oral cavity that may appear to change over a chronological sequence.

### BACKGROUND OF THE INVENTION

Examination of the oral cavity of persons, in particular examination of the dental substance and periodontium, are typically performed by placing a person in an almost horizontal position and illuminating the oral cavity diffusely by means of a special light source. The teeth illuminated in this way are observed and evaluated by the treating dentist. Observation of the teeth and the periodontium are optionally performed using a small mirror, which can be placed in the oral cavity by the dentist by means of a handle section.

With the observation of the oral cavity of a patient described here, there is the problem that the examination results depend greatly on the prevailing illumination conditions, the handling of the patient and also the daily condition and subjective attitude of the dentist responsible for the examination. Documentation of the examination, in particular the creation of relevant and comparable photographs, also poses problems in practice.

The present invention provides solutions to the above-noted problems and allows for an especially effective observation of the oral cavity of a patient by an operator. The present invention also provides for an especially informative documentation of the progress of treatment or the changes in the dental substance and periodontal substance over time.

More specifically, the benefits can be achieved by providing a device for observation of the oral cavity, including a mirror arrangement having at least one mirror element assigned to a certain observation zone in the area of the oral cavity, and a positioning structure for positioning the mirror arrangement in the oral cavity in a reproducible manner.

In this way, it is advantageously possible to perform a photographic observation of the area of the oral cavity of a patient over an extended period of time, such that the images thereby generated can be compared with one another. The device of the present invention is suitable for photographic detection of plaque on a person's teeth and thus for comparing the efficacy of dental treatment agents by means of comparative digital photographs, in particular of the lingual surface of the teeth facing the oral cavity.

Further, images generated by the invention can be used in evaluation of the teeth on the basis of proven schematics, e.g., the so-called Turesky Modified Plaque Index. Through the concept, it is possible to generate in particular digital images by means of a “mirror bridge” coordinated individually with the respective person to be examined, whereby this mirror bridge is placed on the patient's teeth at suitable intervals of time with a high reproducibility from one session to the next, creating essentially uniform recording conditions, or it can be otherwise attached to the teeth temporarily with precise positioning data with a high reproducibility.

### SUMMARY OF THE INVENTION

A device for observation of the oral cavity that comprises a mirror arrangement having at least one mirror element assigned to a certain observation zone in the oral cavity, and a positioning structure for positioning the mirror arrangement in the oral cavity in a reproducible manner.

### BRIEF DESCRIPTION OF THE DRAWINGS

Additional details and features of the invention are derived from the following description in combination with the drawings, in which:

FIG. 1 shows a top view of a device for observation of the oral cavity, here for observation of the maxilla;

FIG. 2 shows another diagram of the device according to FIG. 1 to illustrate additional structural details of same;

FIG. 3 shows a view of the device according to FIGS. 1 and 2 as seen from beneath;

FIG. 4 shows a perspective view of the device according to FIGS. 1 to 3 to further illustrate the connection of the mirror elements;

FIG. 5 shows a diagram to illustrate the device according to FIGS. 1 to 4 in the inserted state;

FIG. 6 shows a perspective diagram to illustrate the device in the applied state;

FIG. 7 shows a variant of a device for observation of the mandibular area;

FIG. 8 shows a top view of the device according to FIG. 7;

FIG. 9 shows a diagram to illustrate the arrangement of the device according to FIGS. 7 and 8 in the inserted state;

FIGS. 10 to 12 show additional perspective diagrams to illustrate the device according to FIGS. 7 to 9 in the inserted state;

FIG. 13 shows a basic diagram to illustrate the additional interaction of the device with a camera system;

FIG. 14 shows a diagram to illustrate the relationship of the individual mirror elements with certain dental areas;

FIG. 15 shows a diagram to illustrate the arrangement of a mandibular variant of the device in combination with the respective camera system;

FIG. 16 shows a diagram to illustrate the correlation of certain mirror elements with certain mandibular tooth areas; and

FIG. 17 shows another diagram to illustrate the arrangement of a device in the mandibular area of a patient.

### DETAILED DESCRIPTION OF THE INVENTION

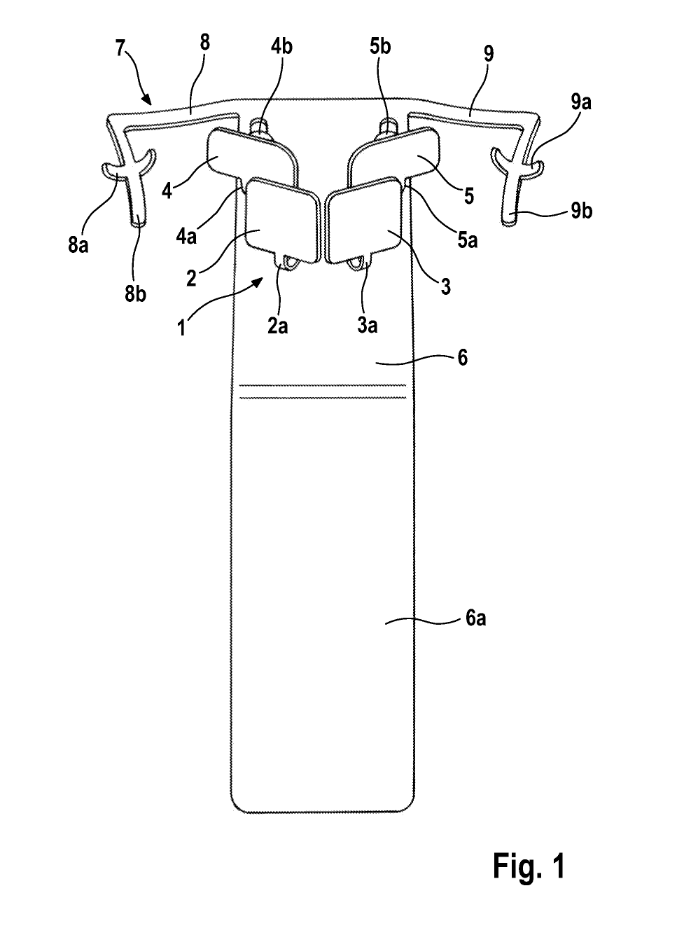

FIG. 1 shows one embodiment of the device of the present invention. This device comprises a mirror arrangement 1 having multiple mirror elements 2, 3, 4, 5, each assigned to a certain observation zone in the patient's oral cavity area. These mirror elements are attached to a base plate 6. The device also comprises a positioning structure 7, which comprises a left positioning arm 8 and a right positioning arm 9 in the exemplary embodiment illustrated here. The positioning structure 7 is designed so that it is adaptable to each patient's specific jaw geometry and thus also allows positioning of the mirror arrangement 1 in the oral cavity in a reproducible manner at longer periods of time.

With the exemplary embodiment shown here, the positioning structure 7 is designed so that the required positioning effect is achieved in cooperation with the patient's teeth. The positioning structure 8 here is provided with plastically deformable arm and brace sections 8a, 8b and/or 9a, 9b. In the exemplary embodiment shown here, the arm and/or brace sections 8a, 8b, 9a, 9b are designed so that they can be adjusted to the geometry of the patient's jaw by means of a bending deformation that is performed once as part of the main adjustment.

It is also possible to provide additional structures that serve the purpose of reproducible positioning of the mirror arrangement, optionally positioning elements formed by molding compounds, on the positioning structure 7, in particular in the end area of the arm and brace sections 8a, 8b, 9a, 9b. Such positioning elements may be adjusted here to the local geometry of the teeth. In the exemplary embodiment shown here, the mirror arrangement is designed so that the alignment of the individual mirror elements 2, 3, 4, 5 is adjustably variable so as to optimize the factors for the observation of the respective zones in the area of the patient's oral cavity. The mirror elements can be adjusted, as shown in greater detail in conjunction with the following figures, first by plastic deformation of the fastening neck sections 2a, 3a, 4a, 5a provided here and by temporary loosening and corresponding tightening of fastening screws 4b, 5b.

The base 6 in the exemplary embodiment shown here is designed as an elongated rectangular body. The exterior section 6a of the base 6, which protrudes above the occlusal surface after application of the device in the patient's oral cavity can serve to temporarily secure the jaw and to secure a certain position relative to a recording system, in particular a digital camera and special lighting.

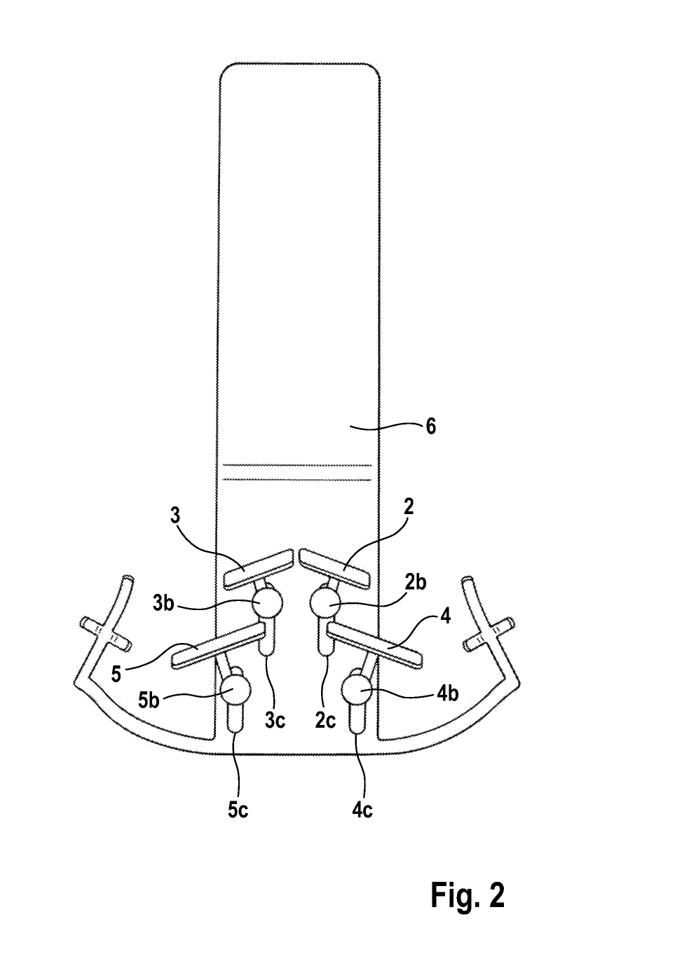

FIG. 2 shows a different view of the device according to FIG. 1 showing the area situated behind the mirror elements 2, 3, 4, 5. As shown in this view, the mirror elements 2, 3, 4, 5 can be secured on the base 6 as needed by means of positioning screws 2b, 3b, 4b, 5b. Therefore, in the exemplary embodiment shown here, elongated boreholes 4c, 5c, 2c, 3c are provided in the base 6.

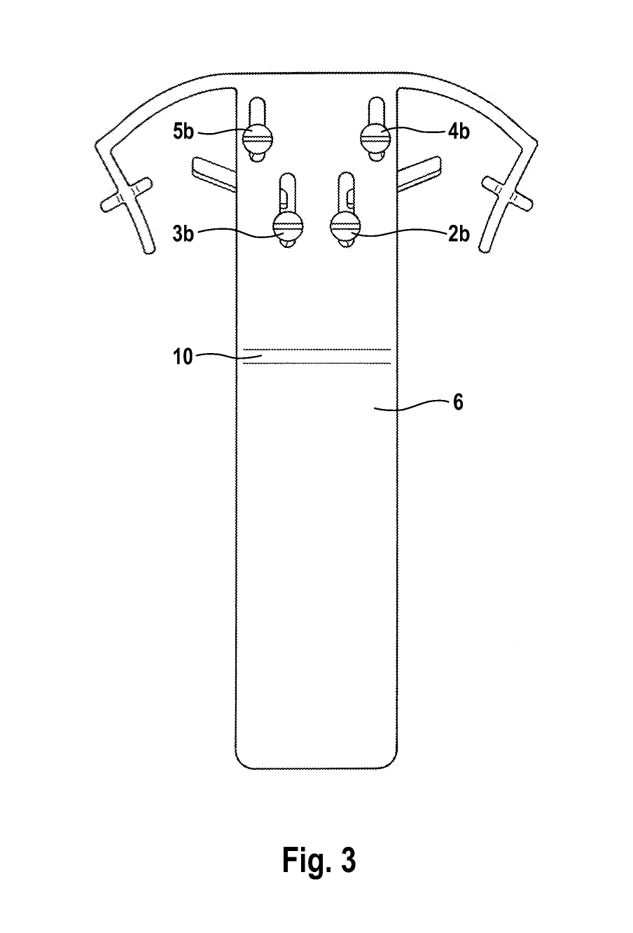

FIG. 3 shows the structure of the device for observation of the oral cavity with a view of the underside of the base 6. In this diagram, the screw heads of the fastening screws 2b, 4b, 3b, 5b are also shown. A beading section 10 may also be formed in the base 6, by means of which the depth of penetration of the base 6 can be defined in combination with the patient's anterior incisors.

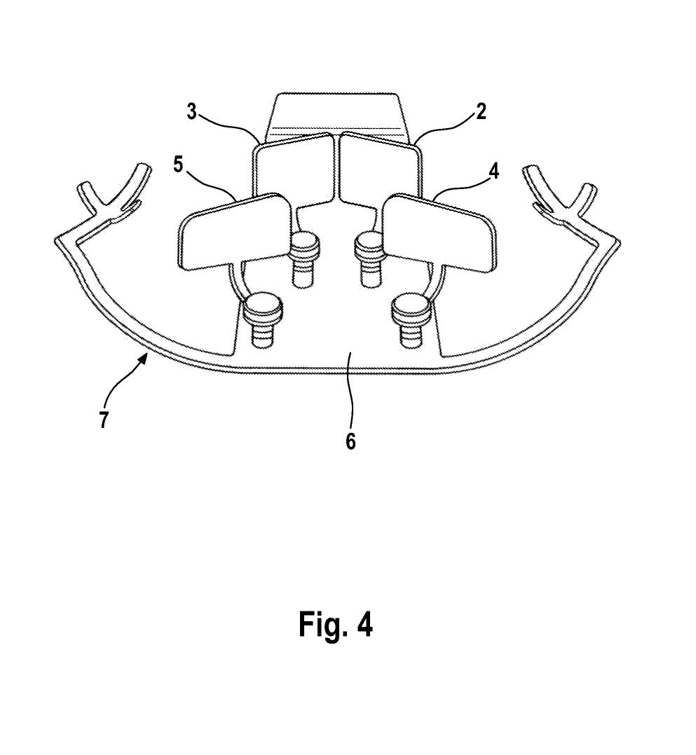

FIG. 4 shows the device of the present invention from another point of view. As is discernible from this view, the mirror elements 2, 3, 4, 5 are secured on the base 6 in an adjustably variable manner. The adjustment of the mirror elements 2, 3, 4, 5 with respect to the base 6 as well as the positioning structure 7, which is defined in the patient's oral cavity, is coordinated only once with the respective patient according to the invention. The configuration of the device achieved in this way is no longer altered during observation processes which take place over longer intervals of time. This achieves the result that the same observation conditions always prevail for all observation processes that follow one another in time. Especially good comparability of the photographic data generated by the device is achieved in this way.

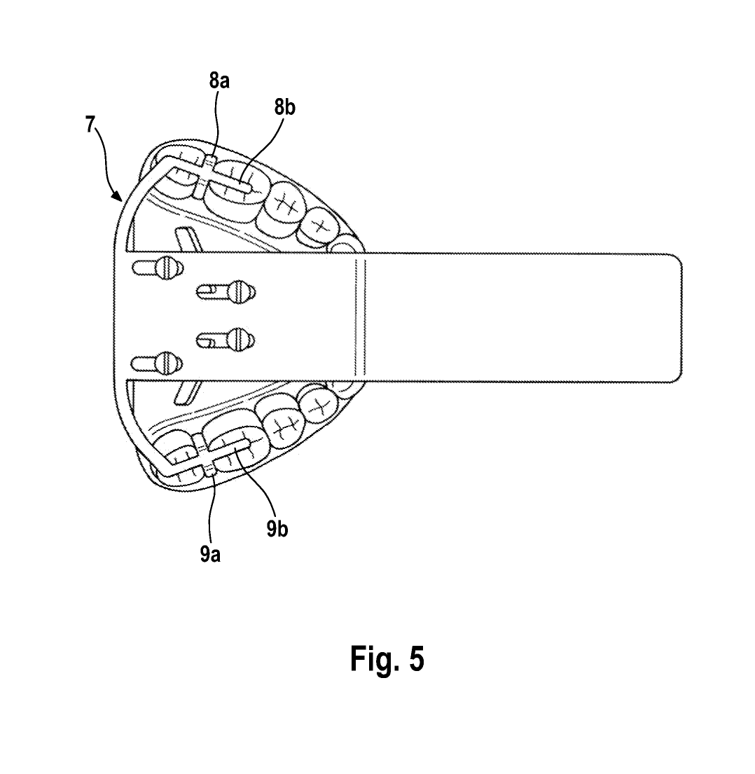

FIG. 5 illustrates how the device of the present invention can be positioned on a patient's maxilla with a high reproducibility. The positioning structure 7 here is coordinated with the geometry of the individual patient's teeth and by appropriate plastic reshaping, so that the arm sections 8b, 9b are positioned interdentally. The brace sections 8a, 9a are bent so that they cause a well-defined positioning of the positioning structure 7 through insertion into the interspaces of the neighboring buccal teeth.

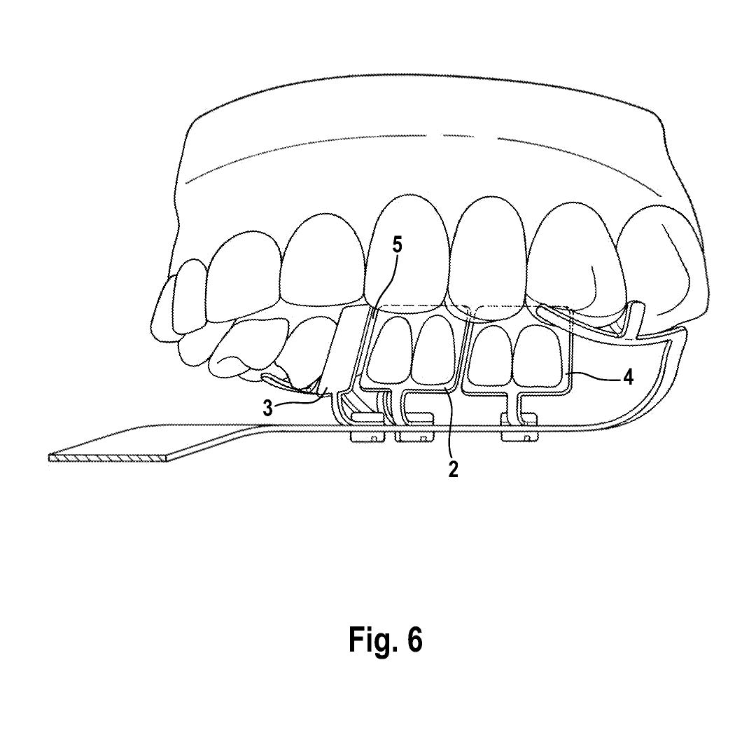

FIG. 6 illustrates in the form of a perspective diagram how the interior surfaces of the maxillary or mandibular teeth facing the lingual space can be observed by using the device for observation of the oral cavity by means of the mirror elements 2, 3, 4, 5 described above and can be photographed under reproducible imaging conditions. The mirror elements 2, 3, 4, 5 are designed so that they each image at least one tooth. The mirror elements 2, 3, 4, 5 are aligned here so that in reproduction of two teeth, the discernible interdental area is situated essentially in the central area of the respective mirror element. This makes it possible to photograph the transitional area between two neighboring teeth in particular.

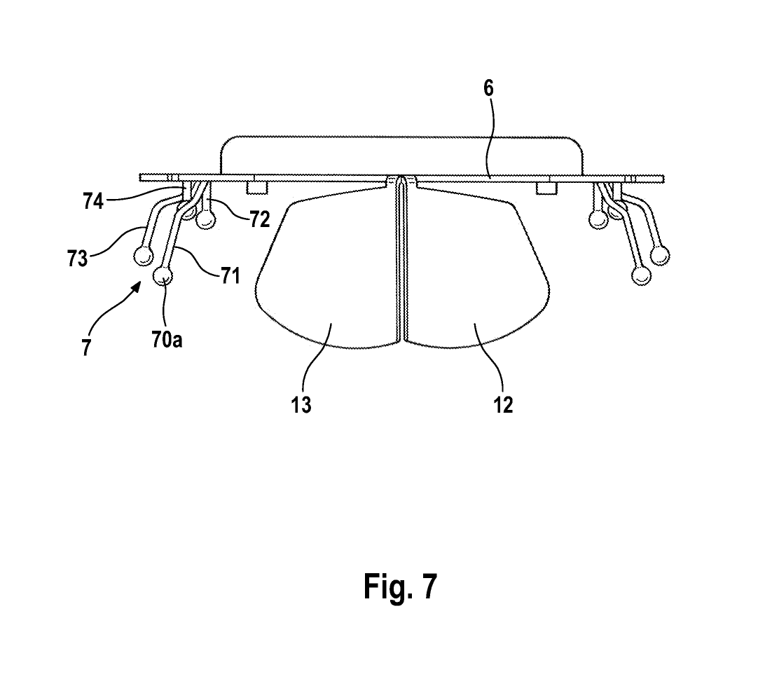

FIG. 7 shows a variant of the device provided for examination of the lingual surfaces. This device also includes a base 6 with the mirror elements 12, 13 mounted on it. In this exemplary embodiment, the positioning structure 7 comprises multiple positioning occlusal stops 71, 72, 73, 74, each being provided with a supporting head 70a. These positioning occlusal stops 71, 72, 73, 74 are coordinated with the geometric properties of the patient's maxillary or teeth and/or soft tissues mandibular within the context of an adjustment step, so that the base 6 is positioned in a defined manner on the patient's dental surfaces with a high reproducibility due to these rententive clasps 71-74. The interior walls of the anterior teeth as well as the cuspids and the premolars can then be recorded photographically by means of the mirror elements 12, 13 that are discernible here. This device variant is suitable for both maxillary and mandibular areas.

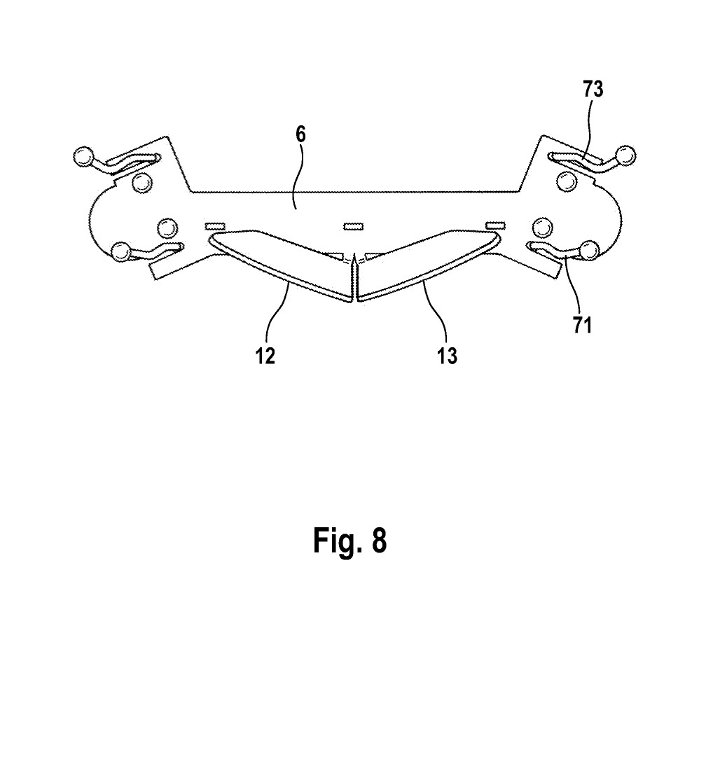

FIG. 8 shows another embodiment of the device according to FIG. 7 which is provided for examination of the mandible. The mirror elements 12, 13 are manufactured as an integral component of the base 6 by means of a punching or cutting operation. The positioning occlusal stops 73, 71 may also be manufactured as an integral component of the base 6 or may be attached thereto, in particular by soldering.

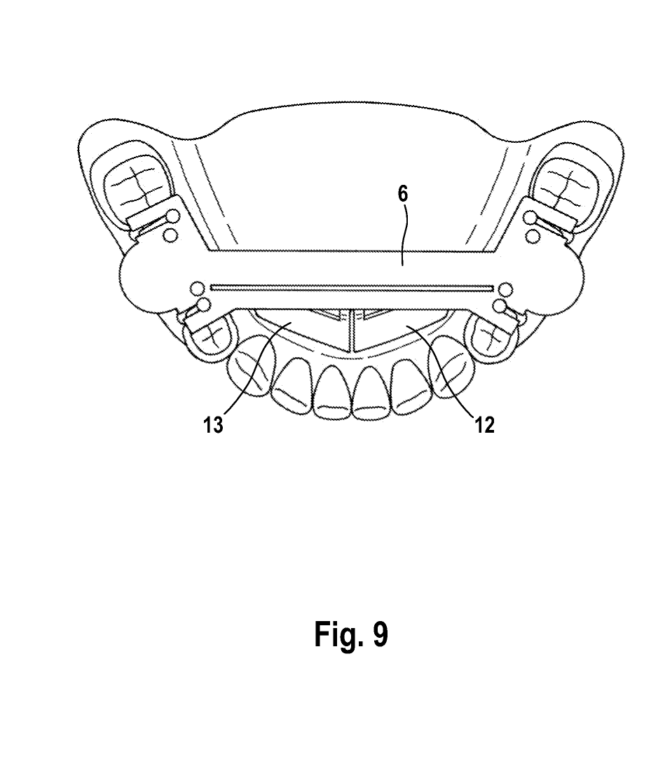

FIG. 9 also illustrates another embodiment of the device of the present invention. As shown in this diagram, the base 6 is designed as a reinforced transverse web, which is held in place with retentive clasps already described above in conjunction with FIGS. 7 and 8. The interior wall areas of the incisors of the mandible as well as the cuspids can be observed and photographed by means of the mirror elements 12, 13.

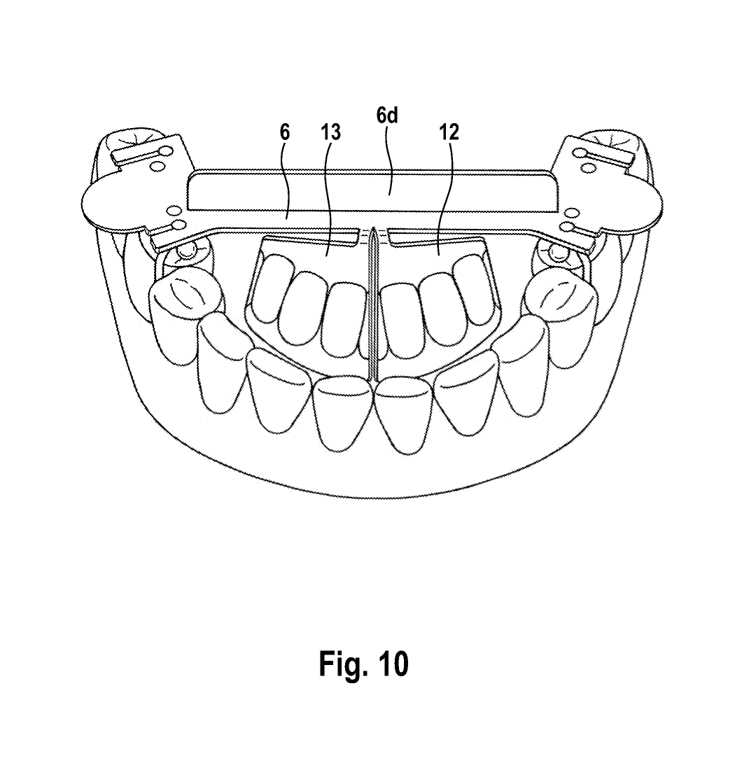

FIG. 10 is a perspective view of the functioning of the device in the form of a perspective diagram. The base 6 which supports the mirror elements 12, 13 is provided with a reinforcing web 6d, as can be seen in this diagram, and serves to facilitate handling. By means of the mirror elements 12, 13, the lingual surfaces of the anterior teeth of the mandible can be visualized and photographed especially well as shown in this diagram. The base 6 is reproducibly positioned above the fissure and the exterior geometry of the premolars of the mandible. There is an extension to allow for insertion and removal of the device.

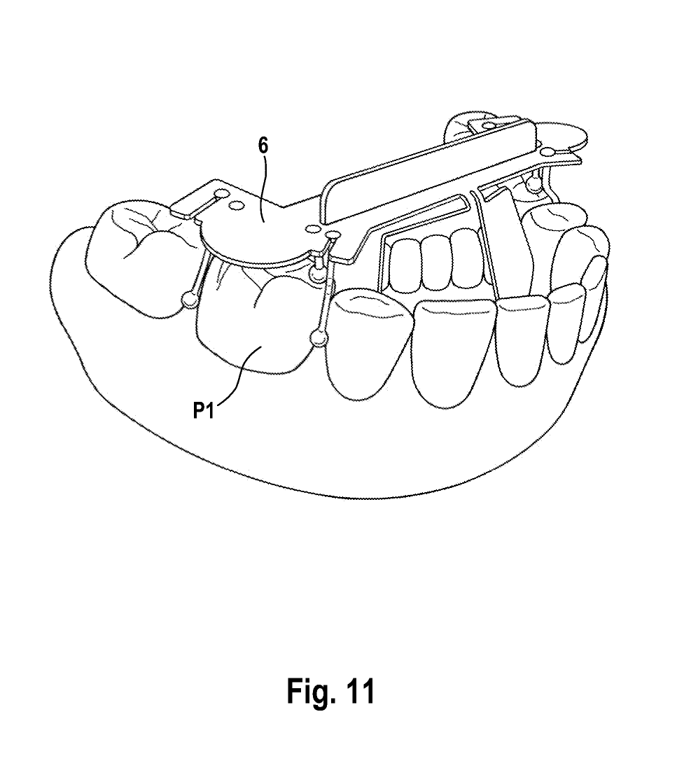

FIG. 11 is another view of the device described above in conjunction with FIG. 10. This diagram shows the positioning of the base 6 on the patient's premolars P1.

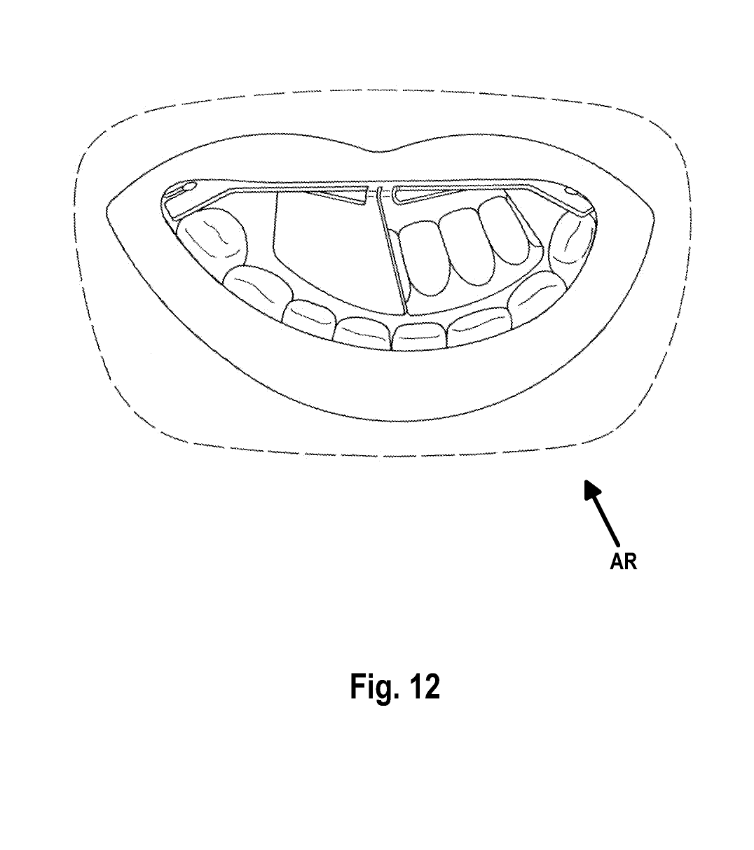

The device may be inserted into the patient's mouth as illustrated in FIG. 12. With the mouth slightly opened and with the lips pulled outward, photographs of the interior wall areas of the mandibular teeth can be created with a high reproducibility and can be evaluated as such, in particular as part of electronic image processing to assess the condition of the teeth and the presence of any deposits. The arrow symbol AR indicates the direction of recording the photograph. In the mirror in FIG. 12, three teeth, the gingival transitional area and several interdental areas are visible in mirror image.

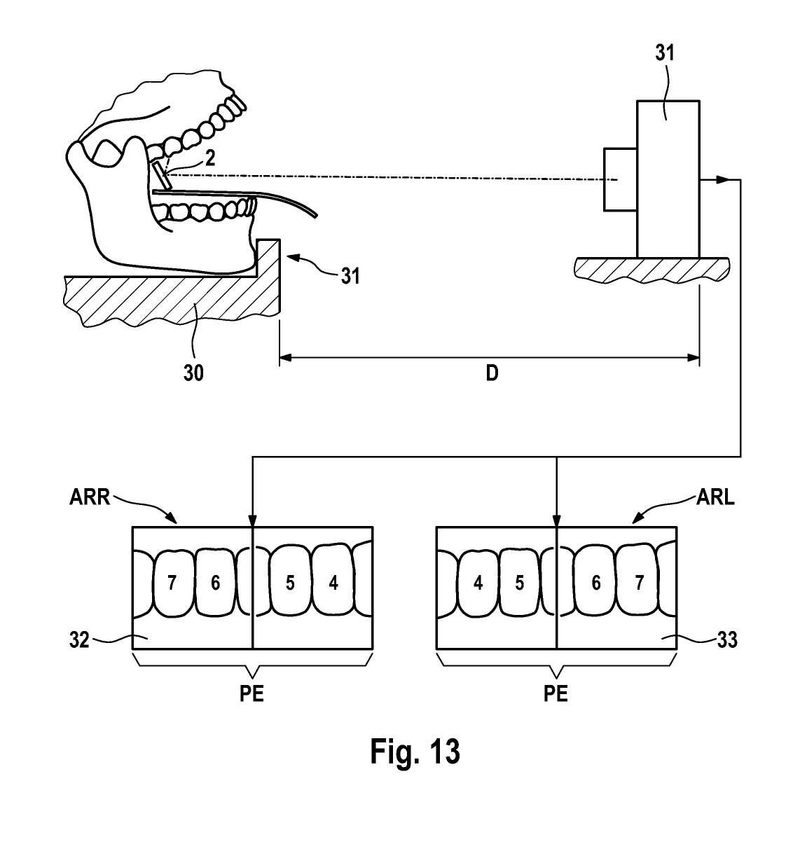

FIG. 13 illustrates in the form of a basic diagram how photographs of the interior of the mouth—namely photographs 32 with optical axis ARR and photographs 33 with optical axis ARL of the maxillary or mandibular areas here—can be produced using a camera, such as digital camera 31 by means of the device. To perform the recording operation, the patient's mandible may be supported on a chin support 30, which is provided with an end post 31. A device is inserted into the patient's oral cavity. The individual mirror elements 2 of this device are aligned so that certain sections of the lingual surfaces of the patient's teeth are imaged in the camera system 31. The mirror elements 2 may therefore be aligned manually as part of a one-time adjusting process. Photograph 32 shows the right side teeth 4,5,6,7 and photograph 33 the left side teeth 4,5,6,7 of the upper jaw.

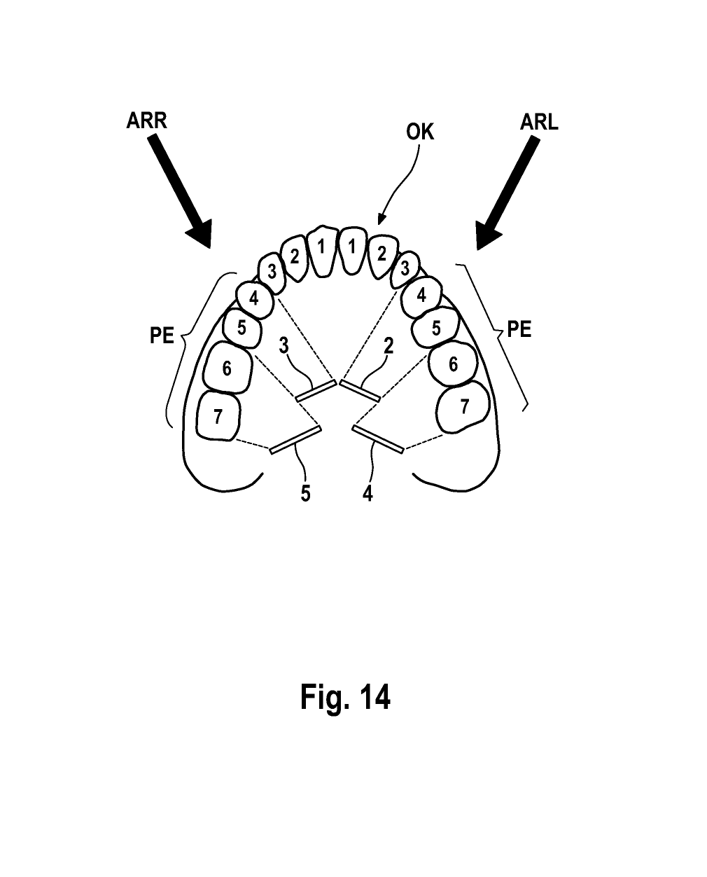

FIG. 14 illustrates how a patient's specific maxillary or mandibular surfaces (upper faw) OK can be imaged by means of the mirror elements 2, 3, 4, 5. The mirror elements 2, 3, 4, 5 may be aligned so that a corresponding photograph of the interior area of the mouth can be made from a central camera position or, as indicated here, from a camera position at the left or right. This camera position opposite the mirror arrangement inserted into the patient's oral cavity can be accomplished either by repositioning the camera or by rotating the patient's head, preferably jointly with the chin support. It is also possible to provide additional fixation and positioning means on the device in the area of the chin support 30 for observation of the oral cavity by means of which, in addition to a reliable reproducible alignment of the device in the patient's oral cavity, a consistent and uniform positioning of the camera 31 with respect to the device for observation of the oral cavity is ensured. The photographs may be created at a distance D, which is defined by the chin support 30 and the camera suspension. Due to the positioning of the mirror elements 2 on the patient's jaw, the distance of the mirror from the teeth is defined on the whole. The arrow symbol ARR symbolizes the optical axis of the camera in photographic imaging of the upper right dental lamina of the maxilla OK. The arrow symbol ARL symbolizes the optical axis of the camera in photographic imaging of the left upper dental lamina. By means of the mirror arrangement shown here, plaque can be detected on the teeth characterized by the numbers 4 through 7. The alignment of the jaw with the optical axis of the camera system may be performed by corresponding pivoting of the head. As an alternative to this, it is also possible to position the camera accordingly.

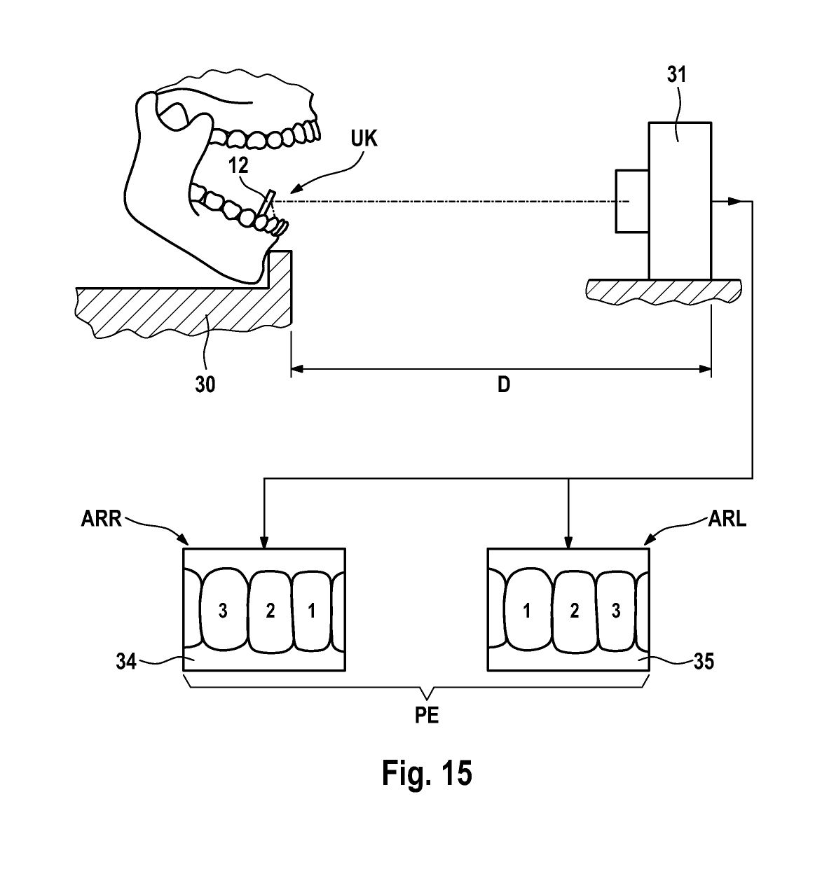

FIG. 15 shows the photographic observation of a patient's lingual surfaces in the form of a schematic diagram. The device is shown mounted on the occusal surfaces of maxillary or mandibular teeth (lower jaw) UK. By means of the mirror element 12, which is visible here, the lingual surfaces of the anterior incisors are reflected into the camera system 31. The device is supported on the chin support. This yields a fixed distance D between the device and the camera system 31. Photograph 34 shows the right side teeth 1,2,3 and photograph 35 the left side teeth 1,2,3 of the lower jaw UK.

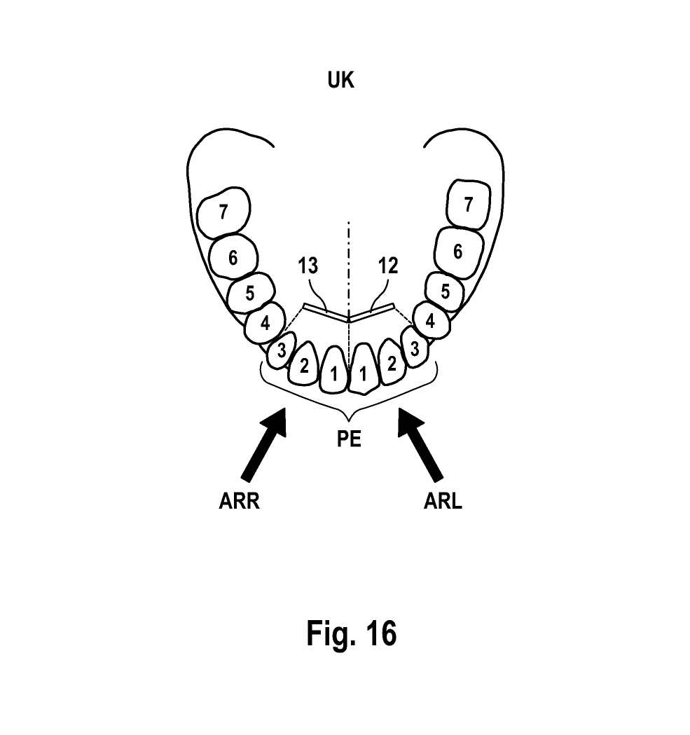

FIG. 16 illustrates in the form of another function diagram how the entire lingual surface area of the anterior incisors of the mandible or maxillary jaw (lower jaw) UK of a patient can be observed via the mirror elements 12, 13. The mirror arrangement illustrated here comprises two mirror elements 12, 13, which serve to detect plaque in the plaque detection area PE—here the lingual surface of the six anterior teeth of the maxillary or mandibular jaws UK.

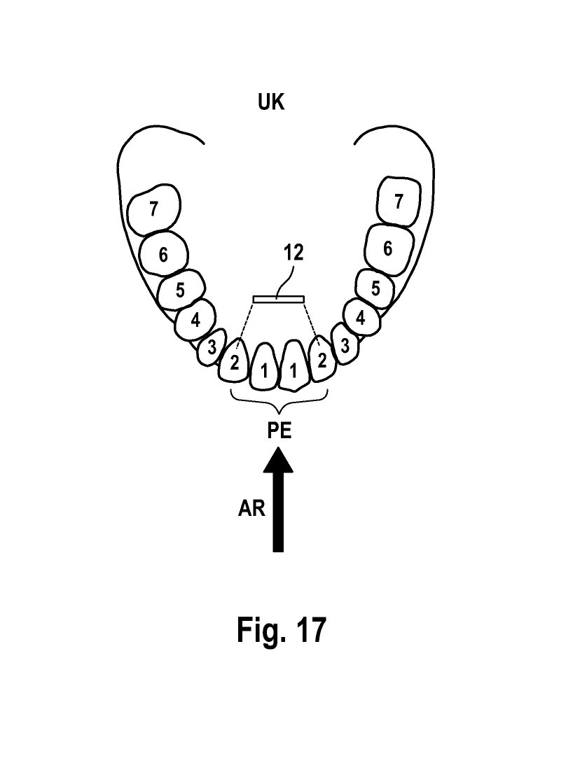

FIG. 17 illustrates a variant of the device in which the interior lingual of the anterior incisors of the jaw UK can be observed by using only one mirror element 12, which is reproducibly positioned on the jaw UK. This arrangement is suitable for detection of plaque in the plaque detection area PE (posterian teeth) indicated here. In the exemplary embodiment illustrated here, this area comprises the four anterior teeth 2, 1, 1, 2 of the jaw UK. To generate corresponding photographs, the mandible is aligned with the optical axis AR of a camera system as shown. The device may be equipped with an aiming device, which facilitates the alignment of the patient's jaw with the optical axis of the camera system.

Both the upper and lower jaws can have two separate devices to view the two distinct areas: anterior and posterior. This results in four devices:

I) upper anterior,

II) upper posterion,

III) lower anterior, and

IV) lower posterior.

According to one embodiment of the invention, the positioning structure is designed so that the positioning effect is achieved in relationship to the patient's teeth. The positioning structure may be designed so that it is positioned reproducibly in particular due to the fissure geometry and optionally also due to other prerequisites pertaining to dental geometry, e.g., with the geometry of the interdental areas determined by neighboring teeth.

The positioning structure may be designed so that it can be coordinated with each patient's specific physiological conditions, i.e., in particular his/her set of teeth and oral cavitygeometry, by using relatively simple tools, in particular bending tools. The positioning structure may therefore comprise wire, brace, rod and/or flat material sections, which can be subjected to plastic deformation with the application of a moderate force and by using conventional dental technical equipment and otherwise reliably retain the shape and geometry imparted to them under otherwise typical loads. The adjusted positioning structure may also be provided with additional reinforcing and securing means even after the initial adjustment, thereby ensuring that this individual adjustment will be retained.

It is also possible to equip the positioning structure with arm sections and strap sections that can be deformed plastically temporarily, i.e., only for the initial adjustment process. In addition, it is also possible to attach seating sections, which may be adjusted to the local dental geometry, for example, to the positioning structure. These seating sections may be formed by temporarily plastifiable or curable compounds, for example.

According to one aspect of the present invention, the device is also designed so that the alignment of the mirror elements is also adjustably variable. The mirror elements may be positioned in particular by means of screw, bending, clamping and joint mechanisms. It is possible in particular to mount the mirror elements on a main carrier device by means of plastically deformable mounting structures.

The mirror elements may be designed as flat surfaces. However, it is also possible to design the mirror surfaces as curved surfaces, in particular with a concave or convex curvature, to impart certain optical imaging properties, in particular an enlargement effect, to the surfaces if necessary. The mirror elements and support sections may be fabricated by laser cutting and CAD/CAM systems.

The mirror elements are advantageously arranged and aligned so that largely overlap-free mirror images are formed in observation from one or more defined observation centers. The mirror arrangement may be embodied so that it can ultimately be used to generate images which reveal the facial surfaces of the anterior teeth as well as, by means of the mirror arrangement, the lingual surfaces of the buccal teeth and optionally also the in at least some sections when the lips are open and the mouth is at least partially open.

The mirror arrangement may be embodied so that these two mirror elements cover not only the lingual surfaces, but two mirror elements may be assigned to cover some orall of the facial surfaces. The mirror arrangement may also be designed so that it comprises a mirror element assigned to any specific area.

The respective mirror elements may be arranged and aligned so as to achieve an extensive utilization of the viewing area defined between the upper and lower dental dimensions (occlusal—gingival). It is also possible to design the mirror arrangement so that individual mirror elements thereof can be brought temporarily into a non-use position and a larger field of vision can be released for the lower mirror elements in this non-use position.

The device may also be designed so that it comprises a variant for observation of the maxilla and an alternative variant for observation of the teeth of the mandible.

The device may be individually, custom-made for each patient and may be used only by this patient at certain intervals and/or from one visit to the next. The device for observation of the oral cavity is suitable for use in combination with digital camera systems.

The device may be designed so that it offers additional positioning systems, which also ensure that a certain position of a recording camera with respect to the mirror arrangement is maintained. Due to the device, changes in the area of the teeth and jaw over time can be recorded with a high reproducibility of the observation positions.

The device is suitable for determining the color of teeth, for detecting the buildup of dental deposits, in particular dental calculus, for evaluating the gingiva and the tooth color. In certain embodiments, it may be possible to ascertain the aforementioned properties in the area of the tooth wall sections facing the lingual or facial surfaces. The mirror arrangement may be made of a corrosion-resistant material, such as, for example, a stainless steel material with or without a rhodium or silver coating by an electroplating process.

In one embodiment, the device comprises, for example, at least one mirror segment which advantageously projects the area of the dental surface facing the lingual or facial space as well as the gingival margin. This individual mirror element or optionally the additional mirror elements may be designed so that they image one or more teeth and provide images of the transitional area between two teeth that are clear.

The device may be designed so that the mirror elements provided by it already have an alignment in a basic setting that takes into account typical usage so that after adjustment of the positioning structure, no corrections in the alignment of the mirror elements are necessary. The positioning structure may also be designed so that it is compatible with common jaw shapes with a high statistical probability.

In addition, it is possible to offer the device in the form of a set comprising different mirror arrangements in particular with regard to the overall size. Small geometric marks may be formed on the individual mirror elements, permitting a reliable determination of the position of the mirror elements within the oral cavity as well as the position of the mirror element with respect to the camera system within the context of analysis of photographs obtained accordingly.

It is possible to provide the device with reference structures, in particular at least one color reference mark for example a standard ceramic shade guide segment that is sufficiently stable with regard to resistance to aging and discoloration. By means of this reference structure mounted on the mirror system, the image reproduction properties of the camera system may be checked, if necessary, and an image calibration may be performed.

In a further advantageous manner, marks, e.g., in the form of a barcode or some other unambiguous pattern may be applied to the device, making it possible to reliably archive the images thereby obtained without any risk of confusion and to correlate them with a person or a test series performed on this person.

The device for observation of the oral cavity can be positioned quickly in the patient's oral cavity with a high reproducibility. The device may be largely prefabricated model and can be adjusted for one test subject with little need for post-processing and adjustment. The device for observation of the oral cavity may be designed so that it can also be subjected to a steam pressure sterilization process (e.g., by an autoclave). The device for observation of the oral cavity may be supplied as a sterile packaged unit, so there is no risk of any microbial transfer.

The image data generated using the device for observation of the oral cavity may be analyzed in an advantageous manner through at least largely automated image processing programs, which provide diagnostic information about certain properties of the oral and/or dental area being examined. Images of the dental and oral cavity area can be generated under special light conditions, including UV light.

With the device, multiple mirror segments aligned individually with regard to their angle of view in a particularly advantageous manner are mounted on a basic structure or a carrier structure. The structure consists of polished corrosion-resistant stainless steel and may comprise a silver or rhodium coating to offer even better reflective properties than corrosion-resistant stainless steel. Mouth opening posts, retaining arms and lateral fastening arms may be incorporated into the device, so that the required distance from the teeth and the special alignment of the mirrors can be recreated quickly and with a high reproducibility. The construction dimensions may be established from a three dimensions (3-D) program.

The alignment angles may be selected so that specially selected areas of the oral cavity as well as the teeth are imaged with a high quality. The mirror arrangement can be used for optical purposes in the visible light range and also in particular in combination with ultraviolet light sources.

The teeth that are photographed may be evaluated by electronic image analysis methods.

### Structure of the Mirror Arrangement

### Maxillary Device

The maxillary device comprises a basic part that functions as the main connecting piece or similar to the mandibular device. This basic element allows simple positioning of the mirror elements and simplifies the overall design of the device. The mirror elements may be manufactured by laser-cutting or any suitable manufacturing method. They may also be attached to the basic part by spot welding, by screw, pinch connections, or any other suitable method and/or means.

Occlusal posts for vertical stability may be provided on the underside of the basic part and hooks for lateral support may also be provided there. These stabilizing elements may be manufactured by special order or may be implemented in the form of retaining hooks and clamps, such as those used in prosthodontics.

### Mandibular Device

The mandibular device may also comprise a basic part which has an L-shaped for H-shaped cross section. This basic part may be designed so that it is responsible for the main stabilization of the mirror arrangement. An extender section may be provided on the basic part, facilitating the insertion and removal of the mirror arrangement and the attachment of same. The mirror segments may be embodied as laser-cut parts, as described above, and may be cut out as parts of the main connecting piece. As an alternative to this, the mirror segments may also be embodied as structures attached by spot welding or otherwise, in particular by screw connection. The occlusal posts extending downward may sit in the fissure and ensure the vertical support. Lateral support may be achieved by neighboring clamps and hooks, which are also provided on the basic part or the main retaining part and are coordinated with the specific geometry of the jaw.

The core of the present invention is essentially to obtain photographic detection of plaque on a person for comparing the efficacy of dental treatment agents by using digital reference photos of the interior surfaces of teeth. The evaluation of the image information may be performed automatically on the basis of the so-called Turesky Modified Plaque Index or by suitable software analysis programs. The photographs may be created according to the invention by means of an individual “mirror bridge” which is adapted specifically to the individual patient and can be attached in a stationary manner (from one visit to the next) either to the teeth (e.g., maxillary bridge) or detachably snapped thereon (example: mandibular bridge) which is reproducible due to uniform recording conditions.

Supporting elements may also be provided on the device to achieve a defined opening of the mouth and, if necessary, to adequately displace the lips.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A device for observation of the oral cavity, comprising:
a mirror arrangement having at least one mirror element assigned to a certain observation zone in the oral cavity, and
a positioning structure for positioning the mirror arrangement in the oral cavity in a reproducible manner.

2. The device of claim 1, wherein the mirror arrangement has at least two mirror elements, each being assigned to a different observation zone in the oral cavity.

3. The device of claim 2, wherein the positioning structure is designed so that the positioning effect is achieved in cooperation with the patient's teeth and the positioning structure can be coordinated with the geometry of the jaw or the patient's hard and soft tissues.

4. The device of claim 1, wherein the positioning structure comprises plastically deformable arm sections and/or brace sections.

5. The device of claim 1, wherein the positioning structure comprises seating sections which are adjusted to optimize for the various factors.

6. The device of claim 1, wherein the alignment of the mirror elements is adjustably variable.

7. The device of claim 1, wherein the mirror elements are attached to the carrying device by means of plastically deformable mounting structures.

8. The device of claim 1, wherein the mirror elements have planar surfaces.

9. The device of claim 1, wherein the mirror elements are arranged and aligned so that they supply largely overlap-free mirror images in observation from a defined observation center.

10. The device of claim 1, wherein the mirror arrangement comprises two mirror elements assigned to the buccal teeth.

11. The device of claim 1, wherein the mirror arrangement comprises two mirror elements assigned to the premolar area.

12. The device of claim 1, wherein the mirror arrangement comprises a mirror element assigned to the incisor area.

13. The device of claim 1, wherein the carrier device can be positioned on the patient's teeth in at least three zones spaced a distance apart.

14. The device of claim 1, wherein the mirror arrangement is supplied in the form of multiple variants for each jaw.
